(19) United States
(12) Patent Application Publication
(10) Pub. No.: US 2001/0023301 A1
(43) Pub. Date: Sep. 20, 2001

(54) Process for the preparation of neotame

(76) Inventors: Wilhelmus H.J. Boesten, Sittard (NL); Peter J.L.M. Quaedflieg, Geleen (NL); Carina S. Snijder, Sittard (NL); Antonius J.J.M Teunissen, Geleen (NL)

Correspondence Address: PILLSBURY WINTHROP LLP, 1600 TYSONS BOULEVARD, MCLEAN, VA, 22102, US

(21) Appl. No.: 09/779,650

(22) Filed: Feb. 9, 2001

Related U.S. Application Data
(63) Continuation of application No. PCT/NL99/00553, filed on Sep. 7, 1999.

(30) Foreign Application Priority Data
Sep. 10, 1998 (NL) 1010063

(51) Int. Cl. C07C 229/00

(52) U.S. Cl. 560/40

(57) ABSTRACT

The invention relates to an improved process for the preparation of neotame by successively

(a) subjecting a mixture of N-benzyloxycarbonyl-L-α-aspartyl-L-phenylalanine-1-methyl ester and 3,3-dimethylbutyraldehyde in solution to hydrogenation in a homogeneous methanolic solvent, in the presence of a hydrogenation catalyst,

(b) separating the catalyst from the solution as a solid substance,

(c) removing a portion, at least, of the organic part of the solvent through evaporation and optionally adding an amount of water before and/or during and/or after that evaporation and

(d) separating the solid neotame formed, optionally after cooling of the system thus obtained, from the remaining liquid and drying it.

The invention relates to an improved process for the preparation of neotame from an aspartame compound and 3,3-dimethylbutyraldehyde under hydrogenating conditions in a solvent.

Neotame is a recently developed, new synthetic, intensive sweetener with a sweetening power which, on a weight basis, is about 10,000× the sweetening power of sugar, and which hence also has a very high sweetening power in comparison with the sweetening powers of other intensive sweeteners so far known. Neotame is for example at least 50× as sweet as aspartame on a weight basis. The chemical structure of neotame corresponds largely to that of aspartame, it being understood that in neotame the free amino group occurring in the aspartyl part of the aspartame molecule has been substituted with a 3,3-dimethylbutyl group. Neotame can be chemically defined as N-[N-(3,3-dimethylbutyl)-L-α-aspartyl]-L-phenyl-alanine-1-methyl ester. Aspartame can be chemically defined as L-α-aspartyl-L-phenylalanine-1-methyl ester, and will also be referred to as APM below.

A process for the preparation of neotame is described in U.S. Pat. No. 5,728,862. In that process an approximately equimolar mixture of aspartame and 3,3-dimethylbutyraldehyde is subjected to hydrogenation in an organic solvent (i.e. a solvent that contains at most 70 wt. % water; the organic solvent is preferably an alcohol, in particular methanol) and in the presence of a hydrogenation catalyst, under suitable conditions in terms of temperature (20-30° C.) and pressure, after which the catalyst is separated from the solution as a solid substance and a water/organic (ratio in the range from 70:30 to 83:17) solvent system is subsequently prepared from the organic phase, from which neotame can be separated via crystallisation.

This method is laborious and time-consuming because aspartame must first be prepared and recovered, and must subsequently be absorbed into an organic solvent for the hydrogenation step. The method consequently demands many process steps and is relatively expensive.

Aspartame is generally prepared either chemically or enzymatically. In the chemical preparation of aspartame use is often made of coupling of an N-protected L-aspartic anhydride, e.g. N-formyl-L-aspartic anhydride, and L-phenylalanine (or the methyl ester thereof). In the (more selective) enzymatic processes for the preparation of aspartame, an N-protected L-aspartic acid derivative, e.g. N-benzyloxycarbonyl-L-aspartic acid, is in practice often coupled with L-phenylalaninemethyl ester. The desired α-coupling product is then formed in a selective manner. In all the processes for the preparation of aspartame the ultimate recovery of the product in a solid form (e.g. through crystallisation, solid/liquid separation and drying, etc.) is a very important part of the overall process.

In other processes for the preparation of neotame so far described the reductive amination step takes place in a solvent system that contains, inter alia, an amount of acetic acid. Such preparation processes (e.g. in U.S. Pat. No. 5,510,508) yield a product that is not pure enough for use as a sweetener in foodstuffs intended for human consumption. Such preparation processes moreover involve substantial deactivation of the employed catalyst, which leads to the consumption of large amounts of catalyst. Such solvent systems are also unattractive from the viewpoint of corrosion of equipment, and effects on the environment.

There is therefore a need for an improved process for the preparation of neotame which can be easily used on an industrial-scale, without the aforementioned drawbacks, in which neotame can be obtained in relatively few process steps, with a favourable amount of catalyst consumption, via a simple hydrogenation step.

It has now surprisingly been found that neotame can be prepared from an aspartame compound and 3,3-dimethylbutyraldehyde under hydrogenating conditions in a highly efficient manner, in very few process steps, namely in only one process step, and without the interim isolation of aspartame, by successively

(a) subjecting a mixture of N-benzyloxycarbonyl-L-α-aspartyl-L-phenylalanine-1-methyl ester and 3,3-dimethylbutyraldehyde in solution to hydrogenation in a homogeneous methanolic solvent, in the presence of a hydrogenation catalyst,

(b) separating the catalyst from the solution as a solid substance,

(c) removing a portion, at least, of the organic part of the solvent through evaporation, and optionally adding an amount of water before and/or during and/or after that evaporation, and

(d) separating the solid neotame formed, optionally after cooling of the system thus obtained, from the remaining liquid and drying it.

In the process according to the invention N-benzyloxycarbonyl-L-α-aspartyl-L-phenylalanine-1-methyl ester (also referred to as Z-APM) is used as the aspartame compound. Wherever this application refers to N-benzyloxycarbonyl (or to Z) this is also understood to be any other protecting group equivalent to the Z protecting group that can be separated through hydrogenolysis, e.g. N-benzyloxycarbonyl groups which contain one or more substituents in their aromatic ring, such as N-p-methoxy-benzyl-oxycarbonyl.

A homogeneous methanolic solvent is in the context of this application understood to be both methanol and homogeneous mixtures of methanol with another solvent miscible with it or with a combination of solvents miscible with it. Such a solvent that is miscible with methanol will of course show inert behaviour under the chosen hydrogenating conditions and relative to the components present in the reaction medium. Examples of such solvents that are miscible with methanol are water, organic solvents such as lower alcohols (C₂-C₄), lower aliphatic ketones (C₃-C₆), e.g. acetone or methyl isobutyl ketone (hereinafter also to be referred to as MIBK), and ethers, e.g. diethylether, in all cases optionally also combined with an amount of water, providing that amount of water does not lead to inhomogeneity of the solvent system.

The homogeneous methanolic solvent is preferably a mixed solvent of methanol and MIBK, and optionally another solvent miscible with it, the solvent most preferably containing 20-95 wt. % methanol, more in particular 45-90 wt. %. Such mixed solvent systems are particularly advantageous because, on the one hand, there will be a homogeneous system under a wide range of hydrogenation conditions and, on the other, solvent combinations of methanol and MIBK are commonly used, or easily obtainable by adding methanol, in enzymatic processes for the preparation of Z-APM. See for example U.S. Pat. No. 5,693,485. In such a case Z-APM does not first have to be isolated and purified before being converted into neotame, but can be converted into neotame directly from the solution in MIBK. Advantages of such a route via Z-APM (in particular also over routes via APM) are first of all that no interim recovery (and optional purification) of APM is required. In addition, the route to neotame via Z-APM clearly involves less formation of by-products and higher yields.

The reaction according to the invention, in which Z-APM is converted into neotame, proceeds excellently in a homogeneous solution. Usually, all the components of the reaction system, except the catalyst, will be present in solution. In the case of high concentrations, one or more of the components may however crystallise somewhat during the reaction, depending on the solvent system used and the temperature of the reaction. Such crystallisation need not be disadvantageous in the process, but will demand additional measures in the upgrading steps to be able to guarantee good separation of the catalyst. The reaction system must for example be heated somewhat first, until all the precipitate formed has dissolved, or an extra amount of methanol has to be added. Such measures can easily be realised by a person skilled in the art.

In general, in the process according to the invention it will be ensured that such an amount of methanol is present during the reaction, and that the reaction temperature is such that no crystallisation of organic product will occur before the catalyst has been separated.

The reaction mixture present during the hydrogenation reaction can be composed in any suitable manner. It is for example possible to first introduce the Z-APM, or a portion thereof, into the solvent system and dissolve it, and then add the catalyst and the 3,3-dimethylbutyraldehyde, and if necessary the rest of the solvent system. It is also possible, as already indicated above, to use product streams in MIBK that become available during enzymatic coupling processes for the preparation of Z-APM, to which methanol may optionally be added, and to subsequently add the catalyst and the 3,3-dimethylbutyraldehyde to it. This also holds when Z-APM is made available in an MIBK product stream via a chemical coupling process.

The 3,3-dimethylbutyraldehyde to be used is commercially available.

Generally, any hydrogenation catalyst known to a person skilled in the art can be used as the hydrogenation catalyst. Preferably use is made of a palladium-on-carbon catalyst. In particular, the palladium-on-carbon catalyst preferably contains 0.1 to 15 wt. % Pd, more in particular the catalyst contains 2-10 wt. % Pd, relative to the catalyst's dry weight. Suitable Pd/C-catalysts are commercially available, e.g. via Engelhard, Degussa or Johnson-Matthey.

The temperature during the hydrogenation will usually be 25-65° C. At a temperature lower than 25° C. the reaction will not, or virtually not, be initiated, at a temperature higher than 65° C. there will be an unnecessarily high risk of the formation of undesired by-products.

The pressure at which the hydrogenation is carried out is usually not very critical. Preferably the hydrogenation step is carried out at atmospheric pressure, with carbon dioxide formed from the Z protecting group immediately being blown down. When the hydrogenation step is carried out at a pressure higher than atmospheric pressure it is preferable to refresh the gas cap (which will come to contain an increasing amount of carbon dioxide during the reaction) with hydrogen gas from time to time. It is less suitable to carry out the hydrogenation step at a pressure lower than atmospheric pressure.

The progress of the hydrogenation reaction can optionally be easily followed via HPLC (high-performance liquid chromatography) analyses of samples taken during the reaction. The hydrogenation step will take approx. 1 to 20 hours, depending on the catalyst chosen (type and amount) and other reaction conditions. This can easily be determined by a person skilled in the art.

The catalyst can be separated from the solution as a solid substance via all the standard techniques for solid/liquid separation known to a person skilled in the art, providing allowance is where necessary made for all the properties of the catalyst used known to a person skilled in the art, such as any pyrophoric properties. After the catalyst has been separated from the otherwise homogeneous reaction mixture, the neotame formed is recovered therefrom. It is preferable to first concentrate the reaction mixture. This will generally be effected through evaporation.

To minimise the formation of by-product, said evaporation will preferably take place at 25-70° C. The best results are obtained when sufficient water to keep the products present in solution is present during the evaporation, and in particular shortly before any crystallisation could take place. That amount of water can easily be determined by a person skilled in the art. A rule of thumb is that the amount of water is so high that all the neotame formed in the reaction is still entirely soluble at the temperature of the evaporation. Extra water will therefore optionally be added during the evaporation. As already mentioned, water is added preferably while a homogeneous solution is still present, i.e. before any crystallisation of neotame occurs. It is particularly advantageous to add water if the solvent system also contains MIBK. In that case the water present also plays a part in the azeotropic and complete removal of MIBK.

Water is preferably added in an amount such that about 50 to 500 wt. % water, relative to the total original amount of organic matter, that is, the total amount of organic solvent and employed organic products, is added.

The organic solvent removed through evaporation can be used again in the process for the preparation of neotame.

The neotame crystallises as a white crystalline compound during or after the evaporation. Preferably an amount of water is added such that the neotame does not yet crystallise during the evaporation, but crystallises only after all the organic solvent has been removed; more in particular the crystallisation of neotame preferably takes place only after cooling from the temperature level during the evaporation to a (lower) temperature in the range from 40 to 0° C.

In a special embodiment of the present invention, namely that in which the hydrogenation reaction is carried out in a mixture of methanol and MIBK (and optionally a little water), after, optionally with the addition of more water, methanol has been removed, an azeotropic mixture of water and MIBK is removed through distillation, and extra water may optionally be added in the last phases of the evaporation to remove all the MIBK. Complete removal of the organic part of the solvent is preferable.

After the crystallisation of neotame (and optionally further cooling of the crystallisation system) the solid neotame obtained can be separated via any technique known to a person skilled in the art, e.g. by means of filtration or centrifugation. After the separation the neotame obtained can optionally be washed, preferably with cold water, and optionally recrystallised. The neotame thus obtained, optionally washed and/or recrystallised, can be dried in any way known to a person skilled in the art. The drying temperature is however preferably not chosen to be higher than 80° C. in view of the risks of decomposition and/or the formation of by-product. Drying can optionally be effected at lowered pressure.

The invention will now be further elucidated with reference to some examples and comparative examples, without being limited in any way by the way in which the experiments have been carried out.

The concentrations of known and unknown components in samples taken at different times, or in the end products obtained, were each time determined by means of elution high-performance liquid chromatography (HPLC). In all the HPLC determinations use was made of a column, measuring 250×3 mm, packed with Inertsil ODS 5 μm, at an oven temperature of 40° C. The following eluants were used: solvent A=10 mM H₃PO₄, solvent B=acetonitrile. At t=0 min. the composition was: 98% A and 2% B; at t=35 min.: 10% A and 90% B. The run time was each time 40 minutes, at a flow rate of 1.2 ml/min. and an injected volume of 20 μl. A photometric UV detector was used for the detection at 210 and 257 nm. All the samples were incorporated in a mixture of 50% methanol and 50% aqueous phosphate buffer, 0.05 M and pH 3.

The samples were taken and analysed in ways known to a person skilled in the art.

### EXAMPLE I

Preparation of neotame from Z-APM in methanol at 40° C.

42.8 g of Z-APM (100 mmol) was dissolved in 500 ml of methanol in a glass 3-liter reaction vessel fitted with a hydrogen dosage device, a stirrer and a drain pipe. 1 g of 5 wt. % Pd/C (which contains 50 wt. % water) and 10 g (100 mmol) of 3,3-dimethylbutyraldehyde were added. The reactor was inertised with the aid of N₂, after which the nitrogen was replaced by 18 l of H₂/hour. The whole was heated to 40° C., after which the reaction started. After 9 hours the reaction was stopped. The catalyst was removed through filtration. The solution was concentrated through evaporation using the Rotavapor at 40° C., at lowered pressure, to approx. 100 ml, after which so much water was added that a precipitate began to form. The mixture was heated to 50° C., which led to the formation of a clear solution. The solution was subsequently cooled to 10° C., after which the neotame crystallised as a white crystalline product. The solid product was separated via filtration and washed using, successively: 30 ml of water and 4×50 ml of heptane. The product was subsequently dried in air at room temperature overnight. 34 grams of the product was obtained, which had a neotame content (determined via HPLC) of 87% (at least 10% of the remaining 13% being present as water). This corresponds to a yield of 78% neotame relative to the amount of Z-APM used.

### COMPARATIVE EXAMPLE A

Preparation of neotame from aspartame in MeOH at 40° C.

29.4 g (100 mmol) of aspartame (APM) was dissolved in 500 ml of methanol as described in Example I 1 g of 5 wt. % Pd/C (which contains 50% water) and 12 g (120 mmol) of 3,3-dimethylbutyraldehyde were added. The reactor was inertised with the aid of N₂, after which the nitrogen was replaced by 18 l of H₂/hour. The whole was heated to 40° C., after which the reaction started. After 9 hours the reaction was stopped. HPLC analysis of the solution revealed a degree of conversion of 98%. The reaction mixture was not upgraded.

Table I below shows the amounts of by-products formed in the example and the comparative example. In addition to neotame (the main product), a few known components (namely: demethylated neotame, referred to as Neo-AP; APM; the diketopiperazine of APM, referred to as DKP-APM; and residual Z-APM) and unknown components (Comp. A with a retention time of 12.7 minutes; Comp. B with a retention time of 29.1 minutes) were found to be present. The corresponding peak areas are shown in the table, and the concentrations (in wt. %) of the known compounds.

TABLE I

| | Reaction | | Comp. A | | | | | Comp. B | |
|---|---|---|---|---|---|---|---|---|---|
| | time | | ret. | DKP- | | | | ret. | |
| Ex./Comp. ex. | (hours) | Neo-AP | 12.7 min | APM | APM | Neotame | Z-APM | 29.1 min. | water |
| Ex. I | | | | | | | | | |
| Peak area HPLC | 9 | 213 | 138 | 76 | 607 | 12579 | 145 | 162 | — |
| Conc. (wt. %) of | | | | | | | | | |
| isolated product | | 1.34 | n.d. | 0.2 | 0.56 | 87 | <0.01 | n.d. | 4.9 |
| Comp. | | | | | | | | | |
| Ex. A | | | | | | | | | |
| Peak area HPLC | 8.5 | 28 | 95 | 61 | 27 | 12633 | — | 1303 | — |
| Content in sal. | | | | 0.03 | 0.12 | 8 | | | |
| (%) | | | | | | | | | |

n.d. = not determined

Table I shows that more known by-products (e.g. Neo-AP) are produced in the reaction in which Z-APM is used as a starting material. Far more unknown by-products (in particular Comp. B) are produced in the comparative reaction, in which APM is used as a starting material.

### EXAMPLE II

Preparation of neotame from Z-APM in methanol at 60° C.

42.8 g of Z-APM (100 mmol) was dissolved in 500 ml of methanol as described in Example I 1 g of 5 wt. % Pd/C (contains 50% water) and 12 g (120 mmol) of 3,3-dimethylbutyraldehyde were added. The whole was heated to 60° C. The reactor was inertised with the aid of N₂, after which the nitrogen was replaced by 18 l of H₂/hour. The reaction was stopped after 9 hours (100% conversion according to HPLC determination). The catalyst was removed through filtration. The whole was evaporated in the wetted-wall evaporator at 40° C. and slightly lowered pressure. A white powder was obtained (41 g, with a neotame content of 88% according to HPLC analysis). The neotame yield was hence 95%, relative to the amount of Z-APM used. See Table II for data on the purity of the product recovered.

### COMPARATIVE EXAMPLE B

Preparation of neotame from APM in methanol at 60° C.

29.4 g of APM (100 mmol) was dissolved in 500 ml of methanol as described in Example I 1 g of 5 wt. % Pd/C (contains 50% water) and 12 g (120 mmol) of 3,3-dimethylbutyraldehyde were added. The whole was heated to 60° C. The reactor was inertised with the aid of N₂, after which the nitrogen was replaced by 18 l of H₂/hour. The reaction was stopped after 9 hours (100% conversion according to HPLC determination). The catalyst was removed through filtration. The whole was evaporated until dry in the wetted-wall evaporator. 37.4 g of white powder was isolated. The neotame yield was 75%, relative to the amount of APM used, with due allowance for the by-products that were also formed.

Table II shows the amounts of (by-)products formed in the above example (after a reaction time of 360 minutes) and in the comparative example (after a reaction time of 305 minutes), based on the peak areas in the HPLC chromatograms obtained. In addition to neotame (main product), a few known components (namely Neo-AP; APM; and DKP-APM; no residual Z-APM) and unknown components (Comp. A, Comp. B, Comp. C. and Comp. D with retention times of 12.7 minutes, 29.1 minutes, 19.1 minutes and 19.8 minutes, respectively) were found to be present. The table indicates only the relevant peak areas; no estimates of the corresponding contents are given.

TABLE II

| | | Comp. A | | | | Comp. C | Comp. D | | |
|---|---|---|---|---|---|---|---|---|---|
| | | ret. | | | | ret. | ret. | Comp. B | |
| Ex./ | | 12.7 | DKP- | | | 19.1 | 19.8 | ret. | |
| Comp. ex. | Neo-AP | min. | APM | APM | Neotame | min. | min. | 29.1 min. | water |
| Ex. II | | | | | | | | | |
| Peak area | 39 | 189 | 104 | 187 | 8943 | 9 | 82 | 595 | — |
| HPLC | | | | | | | | | |
| Content | 0.18 | n.d. | 0.94 | 0.49 | 88.1 | n.d. | n.d. | n.d. | 0.76 |
| (%) | | | | | | | | | |
| Comp. | 33 | 151 | 70 | 23 | 8097 | 3 | 272 | 1201 | — |
| Ex. B. | | | | | | | | | |
| Peak area | | | | | | | | | |

It can be concluded that a higher yield of neotame is obtained from Z-APM (95%) than from APM (75%) in experiments that are otherwise the same. In addition, more unknown by-products (especially Comps. B and D) are formed from APM.

### EXAMPLE III

Preparation of neotame from Z-APM in MIBK:MeOH=1:1

42.8 g (100 mmol) of Z-APM was dissolved in 350 ml of methyl isobutyl ketone (MIBK) as described in Example I. The mixture was heated to 40° C. 350 ml of methanol, 12 g of 3,3-dimethylbutyraldehyde (120 mmol) and 6 g of a 10 wt. % Pd/C catalyst (contains 50% water) were added. The solution was sampled 2×, after 165 minutes (sample 1) and 315 minutes (sample 2). 18 l of H₂/hour was passed through for 5.5 hours. The reaction was stopped. The catalyst was removed through filtration, the solution was weighed (525 g) and analysed with the aid of HPLC (see Table III for the results; the data given therein for sample 3 are the values of the solution obtained after filtration). The analytical yield (calculated) corresponds to a yield of 90% relative to the amount of Z-APM used. An amount of 34.1 g of neotame can thus be obtained after upgrading according to the methods described above.

TABLE III
(All the results are expressed in wt. %):

| Sample | | DKP- | | | |
|---|---|---|---|---|---|
| at time | APM | APM | Neo-AP | Neotame | Z-APM |
| 1 165 min. | 0.095 | 0.006 | 0.013 | 6.45 | <0.01 |
| 2 315 min. | 0.047 | 0.006 | 0.013 | 6.28 | <0.01 |
| 3 315 min. | 0.091 | 0.008 | 0.35 | 6.50 | <0.01 |

### COMPARATIVE EXAMPLE C

Preparation of neotame from APM in MIBK:MeOH=1:1

29.4 g (100 mmol) of APM was added to 350 ml of MIBK in the same way as described for Example III. 350 ml of methanol was added and the mixture was heated to 40° C. The APM did not all dissolve. 12 g of 3,3-dimethylbutyraldehyde (120 mmol) and 6 g of a 10 wt. % Pd/C catalyst (contains 50% water) were added. 18 l of H₂/hour were passed through for 5.5 hours, which resulted in a clear solution. The solution was sampled 2×, after 165 minutes (sample 1) and 315 minutes (sample 2). After the reaction had stopped, the catalyst was removed through filtration; the solution was weighed (581 g) and analysed with the aid of HPLC (see Table IV for the results; the data given therein for sample 3 relate to the 581 g solution). The analytical yield (calculated) corresponds to a degree of conversion of 81% relative to the amount of Z-APM used. An amount of 30.8 g of neotame could hence be obtained after upgrading according to the methods described above.

TABLE IV
(All the results are expressed in wt. %):

| Sample at | | | | | |
|---|---|---|---|---|---|
| time | APM | DKP-APM | Neo-AP | Neotame | Z-APM |
| 1 165 min. | 0.116 | 0.016 | 0.027 | 5.30 | <0.01 |
| 2 315 min. | 0.072 | 0.015 | 0.029 | 5.20 | <0.01 |
| 3 315 min. | 0.020 | 0.016 | 0.028 | 5.30 | <0.01 |

Table V below shows the amounts of by-products formed in the above example and comparative example as peak areas in the HPLC chromatogram.

TABLE V

| | Re- | Comp. A | | Comp. C | Comp. D | Comp. B |
|---|---|---|---|---|---|---|
| Ex./ | action | ret. | | ret. | ret. | ret. |
| Comp. | time | 12.7 | Neo- | 19.1 | 19.8 | 29.1 |
| ex. | (min.) | min. | tame | min. | min. | min. |
| Ex. III | 315 | 79 | 9688 | — | 753 | 36 |
| Comp. | 315 | 61 | 6792 | — | 808 | 34 |
| Ex. C | | | | | | |

More unknown by-products are formed relative to the amount of neotame formed in Comparative Example C (synthesis of neotame from APM) than in Example III (ratios 903/6792 versus 868/9688). The yield from Z-APM to neotame (i.e. 90%) is also higher than that from APM to neotame in the comparative example (81%).

1. Process for the preparation of neotame from an aspartame compound and 3,3-dimethylbutyraldehyde under hydrogenating conditions in a solvent, characterised in that, successively,
(a) a mixture of N-benzyloxycarbonyl-L-α-aspartyl-L-phenylalanine-1-methyl ester and 3,3-dimethylbutyraldehyde in solution is subjected to hydrogenation in a homogeneous methanolic solvent, in the presence of a hydrogenation catalyst,
(b) the catalyst is separated from the solution as a solid substance,
(c) a portion, at least, of the organic part of the solvent is removed through evaporation and an amount of water is optionally added before and/or during and/or after that evaporation, and
(d) the solid neotame formed, optionally after cooling of the system thus obtained, is separated from the remaining liquid and dried.

2. Process according to claim 1, characterised in that the homogeneous methanolic solvent is a mixed solvent consisting of methanol and methyl isobutyl ketone and optionally another solvent that is miscible with them.

3. Process according to claim 1 or claim 2, characterised in that the homogeneous methanolic solvent contains 20-95 wt. % methanol, in particular 45-90 wt. %.

4. Process according to any one of claims 1-3, characterised in that N-benzyloxycarbonyl-L-α-aspartyl-L-phenylalanine-1-methyl ester is made available as a homogeneous solution, obtained in the context of a chemical or enzymatic coupling process, in a solvent system consisting of methylisobutyl ketone to which methanol has been added.

5. Process according to any one of claims 1-4, characterised in that a palladium-on-carbon catalyst is used as the hydrogenation catalyst, in particular a catalyst containing 0.1 to 15 wt. % Pd, in particular containing 2 to 10 wt. % Pd, relative to the dry weight of the catalyst.

6. Process according to any one of claims 1-6, characterised in that the hydrogenation is carried out at a temperature in the range from 25 to 65° C.

7. Process according to any one of claims 1-6, characterised in that the organic part of the solvent is entirely or partially evaporated at a temperature in the range from 25 to 70° C.

8. Process according to claim 7, characterised in that it is ensured that sufficient water to keep the products present in solution is present during the evaporation, in particular shortly before any crystallisation could occur.

9. Process according to claim 8, characterised in that water is added in such an amount that in total about 50-500 wt. % water, relative to the original amount of organic matter, is present before the crystallisation begins.

10. Process according to claim 7 or claim 8, characterised in that all the organic solvent present is removed through evaporation.

11. Process according to any one of claims 1-10, characterised in that solid neotame is obtained through crystallisation at a temperature in the range from 40 to 0° C. and lower than the temperature at which the complete or partial evaporation of the organic part of the solvent takes place.

12. Process for the preparation of neotame as substantially described in the introduction and experiments.
